(19) United States
(12) Patent Application Publication
(10) Pub. No.: US 2022/0081481 A1
(43) Pub. Date: Mar. 17, 2022

(54) USE OF AGENTS CAPABLE OF INDUCING LC3-ASSOCIATED PHAGOCYTOSIS FOR TREATING SUSTAINED INFLAMMATION IN PATIENTS SUFFERING FROM CHRONIC LIVER DISEASE

(71) Applicants: INSERM (INSTITUT NATIONAL DE LA SANTÉ ET DE LA RECHERCHE MÉDICALE), Paris (FR); UNIVERSITE DE PARIS, Paris (FR); ASSISTANCE PUBLIQUE-HÔPITAUX DE PARIS (APHP), Paris (FR); CENTRE NATIONAL DE LA RECHERCHE SCIENTIFIQUE (CNRS), Paris (FR)

(72) Inventors: Sophie LOTERSZTAJN, Paris Cedex 18 (FR); Jinghong WAN, Paris Cedex 18 (FR); Patrice CODOGNO, Paris Cedex 14 (FR); Sanae BEN MKADDEM, Paris Cedex 18 (FR); Renato MONTEIRO, Paris Cedex 18 (FR); Loredana SAVEANU, Paris Cedex 18 (FR); Emmanuel WEISS, Paris (FR)

(21) Appl. No.: 17/422,227

(22) PCT Filed: Jan. 15, 2020
(86) PCT No.: PCT/EP2020/050915

§ 371 Date: Jul. 12, 2021

(30) Foreign Application Priority Data
Jan. 16, 2019 (EP) 19305050.7

(51) Int. Cl. C07K 16/28; A61P 1/16

(52) U.S. Cl. CPC C07K 16/283; A61K 2039/505; A61P 1/16

(57) ABSTRACT

Sustained hepatic and systemic inflammation, in particular originating from monocyte/macrophages, is a driving force for chronic liver disease progression to cirrhosis and underlies the development of multiorgan failure. Therefore, reprogramming mono-cyte/macrophage phenotype has emerged as an interesting strategy to limit inflammation during chronic liver injury. The inventors report here that a non-canonical form of autophagy, LC3-associated phagocytosis (LAP), is endogenously enhanced in blood and liver monocytes from cirrhotic patients and is negatively correlated to the levels of inflammatory markers in these patients. Pharmacological inhibition of LAP components or genetic disruption of LAP (Rubicon-deficient mice in myeloid cells), exacerbates the inflammatory signature in isolated human cirrhotic monocytes and the hepatic inflammatory profile in mice with chronic liver injury, resulting in enhanced liver fibrosis. Mice overexpressing human FcγRIIA in CD11b+ cells show enhanced LAP in response to chronic liver injury, and are protected against inflammation and liver fibrosis. Finally, endogenous activation of LAP is lost in monocytes from severe cirrhotic patients with massive systemic inflammation, and restored upon exposure to intravenous monomeric Immunoglobulin (IVIg). These data shed light on a novel role for LAP in the protection against inflammation during cirrhosis and its progression to severe stages and thus suggest that agents capable of inducing LAP are suitable for treating sustained inflammation in patients suffering from chronic liver disease.

### FIELD OF THE INVENTION

The present invention relates to methods and pharmaceutical compositions for treating sustained inflammation in patients suffering from chronic liver disease.

### BACKGROUND OF THE INVENTION

Chronic liver injury develops in response to alcohol, nonalcoholic steatohepatitis (NASH) or viral hepatitis, and exposes to fibrosis and end-stage cirrhosis^{1,2}. Sustained hepatic inflammation originating from monocytes/macrophages is crucial for progression of chronic liver diseases^{1,3,4} In response to hepatocyte stress, damage and death and to Pathogen-Associated Molecular Patterns, Kupffer cells (the resident liver macrophages), acquire a proinflammatory phenotype. The resulting release of proinflammatory cytokines and chemokines leads to the recruitment of blood monocytes that infiltrate the liver and sustain and perpetuate the inflammatory response, with major deleterious impact on hepatocyte lesions and fibrosis^{1,3}. At advanced stages of the disease, cirrhosis is characterized by severe immune dysfunction and sustained systemic inflammation, arising from increased number of inflammatory monocytes that produce inflammatory cytokines, and drive patients to organ failure (acute-on chronic liver failure, ACLF)⁵. Therefore, there is growing interest in the identification of mechanisms that reprogram monocyte/macrophage phenotype to limit inflammation during chronic liver disease.

Macroautophagy (referred as to autophagy) in macrophages is a master regulator of inflammatory signaling, and controls monocyte differentiation into macrophages and macrophage phenotype^{6,7}. Non-canonical autophagy pathways have also been identified, among which LC3-associated phagocytosis (LAP) has recently emerged as a major mechanism for monocyte/macrophage phenotype reprogramming to an anti-inflammatory phenotype^{8,9}. In particular, LAP controls the autoimmune response by preventing auto-antigen presentation, allowing dead cell clearance and dampening of pro-inflammatory signals. LAP is initiated upon phagocytosis of particles that engage innate immune receptors, such as Pattern-Recognition Receptors (Toll Like Receptors, C-type lectin receptors such as Dectin-1), Ptd-Ser receptors or Fcgamma Receptors, in particular FcγRIIA^{8,9}. This phagocytic process results in the recruitment of some, but not all, members of the autophagic machinery to the stimulus-containing phagosome, facilitating rapid phagosome maturation, degradation of engulfed pathogens, and modulation of the immune response⁸⁻¹⁰. LAP and autophagy are functionally and mechanistically distinct, with major differences including the type of membrane, which is a single membrane-bound structure for the LAP-engaged phagosome, and a double membrane-bound vacuole for the autophagosome. LAP also proceeds independently of the preinitiation AMPK-mTOR-ULK1 autophagy complex, but nevertheless requires some autophagic components, such as the Class III PI3K complex and elements of the ubiquitinylation-like, protein conjugation system (ATG5, ATG7)¹¹. In addition, at early stages of LAPosome formation, The RUN domain containing cystein rich protein Rubicon is needed for producing PI3P from a complex that contains Beclin 1, UVRAG and VPS34 and is also a positive regulator of the phagocytic NOX2 complex, both by binding and stabilizing p22phox, and by promoting assembly of the NOX-2 complex^{8,9,12,13} In contrast, Rubicon inhibits autophagy by preventing maturation of autophagosome¹². The need for Rubicon has therefore allowed to experimentally distinguish the roles of LAP and autophagy in the control of the inflammatory response¹².

The involvement of autophagy in liver inflammation and fibrosis is only emerging. Mice lacking ATG5 in myeloid cells are more susceptible to liver inflammation when exposed to chronic alcohol feeding¹⁴ or chronic toxic injury¹⁵, and show enhanced hepatocyte death and exacerbated fibrosis¹⁵. However the role of LAP in sustained inflammation in patients suffering from chronic liver disease has never been investigated.

### SUMMARY OF THE INVENTION

As defined by the claims, the present invention relates to methods and pharmaceutical compositions for treating sustained inflammation in patients suffering from chronic liver disease.

### DETAILED DESCRIPTION OF THE INVENTION

Sustained hepatic and systemic inflammation, in particular originating from monocyte/macrophages, is a driving force for chronic liver disease progression to cirrhosis and underlies the development of multiorgan failure. Therefore, reprogramming monocyte/macrophage phenotype has emerged as an interesting strategy to limit inflammation during chronic liver injury. The inventors report here that a non-canonical form of autophagy, LC3-associated phagocytosis (LAP), is endogenously enhanced in blood and liver monocytes from cirrhotic patients and is negatively correlated to the levels of inflammatory markers in these patients. Pharmacological inhibition of LAP components or genetic disruption of LAP (Rubicon-deficient mice in myeloid cells), exacerbates the inflammatory signature in isolated human cirrhotic monocytes and the hepatic inflammatory profile in mice with chronic liver injury, resulting in enhanced liver fibrosis. Mechanistically, cirrhotic patients show increased surface and intracellular expression of FcγRIIA and enhanced engulfment of IgG in LC3+ phagosomes that triggers an FcγRIIA/SHP-1-ITAMi anti-inflammatory signaling. In keeping, mice overexpressing human FcγRIIA in CD11b+ cells show enhanced LAP in response to chronic liver injury, and are protected against inflammation and liver fibrosis. Finally, endogenous activation of LAP is lost in monocytes from severe cirrhotic patients with massive systemic inflammation, and restored upon exposure to intravenous monomeric Immunoglobulin (IVIg). These data shed light on a novel role for LAP in the protection against inflammation during cirrhosis and its progression to severe stages.

Accordingly, the first object of the present invention relates to a method of treating sustained inflammation in a patient suffering from a chronic liver disease comprising administering to the patient a therapeutically effective amount of an agent capable of inducing LC3-associated phagocytosis.

As used herein, the term “sustained inflammation” has its general meaning in the art and is used to describe a prolonged inflammatory reaction after an initial activation of the immune system, with an inability to resolve the resultant inflammation.

In some embodiments, the method of the present invention is particularly suitable for the treatment of sustained hepatic inflammation and/or sustained systemic inflammation.

As used herein, the term “hepatic inflammation” refers to the inflammatory response that occur in the liver.

As used herein, the term “systemic inflammation” as used herein refers to an inflammatory response involving major organs or symptoms outside the liver.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

The term “chronic liver disease” is used herein to refer to liver diseases associated with a chronic liver injury regardless of the underlying cause. The chronic liver disease may result, for example, from infectious or autoimmune processes, from mechanical or chemical injury to the liver, or from cancer, all of which are included within the definition of “liver disease.” Chemical injury to the liver can be caused by a variety of toxins, such as alcohol, carbon tetrachloride, trichloroethylene, iron overdose, drug overdose, drug side-effects etc. In some embodiment, the patient suffers from cirrhosis, such as, alcoholic liver cirrhosis and primary biliary cirrhosis (PBC), liver fibrosis, chronic hepatitis, i.e. chronic autoimmune hepatitis, chronic alcoholic hepatitis, non-alcoholic steatohepatitis (NASH, also known as steatosis), viral hepatitis A, B, C, D, E and G, toxic metabolic liver damage, and fatty liver.

In some embodiments, the patient suffers from an alcoholic hepatitis. “Alcoholic hepatitis,” as used herein, includes acute and chronic hepatitis resulting from excessive alcohol consumption, and can range from a mild hepatitis, with abnormal laboratory tests being the only indication of disease, to severe liver dysfunction with complications such as jaundice (yellow skin caused by bilirubin retention), hepatic encephalopathy (neurological dysfunction caused by liver failure), ascites (fluid accumulation in the abdomen), bleeding esophageal varices (varicose veins in the esophagus), abnormal blood clotting and coma.

In some embodiments, the patient suffers from a viral hepatitis. The term “viral hepatitis,” as used herein, refers to hepatitis resulting from hepatitis A, B, C, D, E, or G infection.

In some embodiments, the patient suffers from a non-alcoholic fatty liver disease. As used herein, the term “non-alcoholic fatty liver disease” has its general meaning in the art and is intended to refer to the spectrum of disorders resulting from an accumulation of fat in liver cells in individuals with no history of excessive alcohol consumption. In the mildest form, NAFLD refers to hepatic steatosis. The term NAFLD is also intended to encompass the more severe and advanced form non-alcoholic steatohepatitis (NASH), cirrhosis, hepatocellular carcinoma, and virus-induced (e.g., HIV, hepatitis) fatty liver disease. The term “NASH”, as used herein, collectively refers to the state where the liver develops a hepatic disorder (e.g., inflammation, ballooning, fibrosis, cirrhosis, or cancer), or the state where the liver may induce such a pathological condition, and “NASH” is distinguished from “simple steatosis”; i.e., a condition in which fat is simply accumulated in the liver, and which does not progress to another hepatic-disorder-developing condition.

In some embodiments, the patient has liver fibrosis. “Liver fibrosis” or “hepatic fibrosis” is characterized by the accumulation of extracellular matrix that can be distinguished qualitatively from that in normal liver. Left unchecked, hepatic fibrosis progresses to cirrhosis (defined by the presence of encapsulated nodules), liver and organ failure, and death. In particular, the agent of the present invention is suitable for reducing liver fibrosis.

In some embodiments, the patient has cirrhosis. The term “cirrhosis” is used herein to refer to a pathologic liver condition characterized anatomically by widespread nodules in the liver combined with fibrosis. Cirrhosis represents the final common pathway for must types of chronic liver diseases, including those associated with chronic alcohol abuse, chronic viral hepatitis, metabolic and biliary diseases.

The method of the present invention is particularly suitable for preventing acute-on-chronic liver failure.

As used herein, the term “acute-on-chronic liver failure” refers to acute decompensation of cirrhosis, associated with at least one organ failure and with high short-term mortality rate. With the term “decompensated cirrhosis” is typically meant advanced liver cirrhosis with a range of clinical evidence such as jaundice, ascites, oedema, hepatic encephalopathy, gastrointestinal haemorrhage, portal hypertension, bacterial infections, or any combination.

As uses herein, the term “LC3-associated phagocytosis” has its general meaning in the art and refers to the cellular process wherein elements of autophagy conjugate LC3 to phagosomal membranes. In said process the components of the autophagy machinery thus associate with the phagosome, promoting its fusion with lysosomes (phagosome maturation). Accordingly, the term “agent capable of inducing LC3-associated phagocytosis” refers to any compound natural or not that is capable of inducing said process.

In some embodiments, the agent is a ligand of FcγRIIA. As used herein, the term “FcγRIIA” has its general meaning in the art and refers to a low affinity receptor to the Fc region of immunoglobulin gamma complexes. The encoded protein is involved in the phagocytosis of immune complexes. The term is synonymous to the term “CD32A”.

As used herein the term “ligand” refers to a molecule with the affinity to bind to a second molecule such as a receptor. As used herein the term “bind” indicates that the ligand has affinity for the receptor. The term “affinity”, as used herein, means the strength of the binding of the ligand to the receptor. The affinity of ligand is typically given by the dissociation constant Kd. Preferred methods for determining the affinity of ligands can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of ligands is the use of Biacore instruments.

In some embodiments, the ligand has an Fc region. As used herein “Fc region” includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat et al. (1991, NIH Publication 91-3242, National Technical Information Service, Springfield, Va.). The “EU index as set forth in Kabat” refers to the residue numbering of the human IgG1 EU antibody as described in Kabat et al. supra. Fc may refer to this region in isolation, or this region in the context of an antibody, antibody fragment, or Fc fusion protein. An Fc variant protein may be an antibody, Fc fusion, or any protein or protein domain that comprises an Fc region.

In some embodiments, the ligand is an immunoglobulin. An “immunoglobulin” is a polypeptide that is immunologically reactive with a particular antigen. The term “immunoglobulin,” as used herein, encompasses intact molecules of various isotypes as well as fragments with antigen-binding capability, e.g., Fab′, F(ab′) 2, Fab, Fv and rIgG. See, e.g., Pierce Catalog and Handbook, 1994-1995 (Pierce Chemical Co., Rockford, Ill.); Kuby, J., Immunology, 3.sup.rd Ed., W.H. Freeman & Co., New York (1998). The term also encompasses recombinant single chain Fv fragments (scFv). The term further encompasses bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al., 1993, supra, Gruber et al. (1994) J. Immunol.: 5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301.

In some embodiments, the ligand is a whole IgG immunoglobulin. The term “IgG immunoglobulin” in this context is intended to refer to an immunoglobulin characterized by the presence of a γ (gamma) heavy chain. An exemplary whole IgG immunoglobulin structure comprises a tetramer. Each tetramer is composed of two identical pairs of polypeptide chains, each pair having one “light” (about 25 kDa) and one “heavy” chain (about 50-70 kDa). There are four IgG subclasses in humans: IgG1, IgG2, IgG3 and IgG4. IgG1 and IgG2 are the most common types of IgG. The term “IgG” is further intended to encompass IVIG (intravenous immunoglobulin), SCIG, (subcutaneous immunoglobulin) and IMIG (intramuscular immunoglobulin).

In some embodiments, the patient is administered with a therapeutically effective amount of IVIG. As used herein the term “intravenous immunoglobulin” or “IVIG” refers to a blood product that contains the pooled immunoglobulin G (IgG) immunoglobulins from the plasma of a large number (often more than a thousand) of blood donors. Typically containing more than 95% unmodified IgG, which has intact Fc-dependent effector functions, and only trace amounts of immunoglobulin A (IgA) or immunoglobulin M (IgM), IVIGs are sterile, purified IgG products used in treating certain medical conditions. Although the word “intravenous” typically indicates administration by intravenous injection, the term “IVIG” or “IVIG composition” as used in this patent application also encompasses an IgG composition that is formulated for administration by additional routes, including subcutaneous or intranasal administration.

In some embodiments, the ligand is an anti-FcγRIIA F(ab′)2 fragment. As used herein, the term “F(ab′)2” relates to an IgG fragment consisting of two Fab fragments connected to one another by disulfide bonds. As used herein, the term “Fab” relates to an IgG fragment comprising the antigen binding region, said fragment being composed of one constant and one variable domain from each heavy and light chain of the antibody.

A “therapeutically effective amount” refers to an amount effective of the agent, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of agent are outweighed by the therapeutically beneficial effects. The efficient dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above.

Typically, the agent of the present invention is administered to the subject in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulo se, polyacrylates, waxes, polyethylene-polyoxypropylene-block polymers, polyethylene glycol and wool fat. For use in administration to a subject, the composition will be formulated for administration to the subject. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of the inhibitor of the invention.

The invention will be further illustrated by the following figures and examples. However, these examples and figures should not be interpreted in any way as limiting the scope of the present invention.

### FIGURES

FIG. 1: Inhibition of LAP components enhances the inflammatory signature in blood monocytes from patients with cirrhosis. (A). Inverse correlation between circulating C-Reactive Protein (CRP) levels and the number of LC3+ pHrodo+ phagosomes containing E. Coli bioparticles in blood monocytes from patients with cirrhosis (n=14). *p=0.005, r=−0.7 for LC3+ pHrodo+ phagosomes number vs CRP. (B). PBMC were incubated for 4 hrs at 37° C. with either vehicle, 10 mM 3-MA, 10 μM DPI, 30 μM N8 or control mutant N8W9A peptides Percentage of IL8+ production in CD14+ cells from patients (n=8) and healthy donors (n=6) PBMC. (C). RT-PCR analysis of inflammatory gene expression in blood monocytes from healthy donors (n=6-8) and cirrhotic patients (n=6-11). Results are expressed as fold over respective control. *p<0.05, **p<0.001 for healthy vs cirrhotic. Statistical analysis was performed using Spearman correlation test (A), Mann-Whitney (b, for comparison between healthy and cirrhosis) and Wilcoxon matched-pairs signed rank test (B, for treatment).

FIG. 2: LAP is increased in monocytes from human or mice cirrhotic livers and protects against hepatic inflammation and fibrosis. (A). Human intrahepatic monocytes were isolated from liver explants of control non-tumor or cirrhotic livers (left). Quantification of the number of LC3+ pHrodo+ phagosome containing E. Coli bioparticles in intrahepatic monocytes from control (n=5), or cirrhotic (n=4) human livers, and livers from C57B16/J mice exposed to vehicle (n=2) or CCl₄ (n=5) for 7 weeks. Quantification was performed on at least 40 cells from 15 fields/individual. 63× Magnification, scale bar: 5 μm. **p<0.001 for control or vehicle vs cirrhotic liver. (B). Rubicon^{Mye-/-} mice and their WT littermates were exposed to CCl₄ for 7 weeks. Quantification of inflammatory infiltrate in Hematoxylin and Eosin stained liver tissue sections (20× magnification, scale bar: 100 μm). Hepatic inflammatory gene expression (C) and hepatic cytokine production (D) by RT-PCR and ELISA analysis. (E). Representative quantification of sirius red staining and α-SMA immunostaining (F). 10× magnification, scale bar:200 μm. *p<0.05 for Rubicon^{Mye-/-} (n=4) vs Rubicon^{flox/flox} (n=6). Statistical analysis was performed using Mann-Whitney.

FIG. 3: FcγRIIA-mediated activation of LAP as a potential therapeutic target. (A). hFcγRIIA-Tg mice (n=7) and WT littermates (n=6) were exposed to CCl₄ for 10 weeks. Quantification of the number of LC3+ pHrodo+ phagosomes containing E. Coli bioparticles in intrahepatic monocytes on at least 40 cells from 15 fields/mouse liver. 63× Magnification, scale bar: 5 μm. **p<0.001 for hFcγRIIA-Tg vs WT littermates. (B). Quantification of inflammatory infiltrate in Hematoxylin and Eosin stained liver tissue sections (20× magnification, scale bar: 100 μm). ELISA of hepatic IL6 and IL1-β. *p<0.05 and **p<0.001 for hFcγRIIA-Tg vs WT littermates. (C). Quantification of sirius red staining and α-SMA immunostaining (10× magnification, scale bar: 200 μm). *p<0.05 for hFcγRIIA-Tg vs WT littermates. (D). MFI of surface FcγRIIA expression in CD14+ PBMC from ACLF patients (n=7) and healthy donors (n=6) by flow cytometry. Results from cirrhotic patients (dashed lines) were included for comparison. *p<0.05 for ACLF vs healthy or vs cirrhosis. (E). Quantification of the number of LC3+ pHrodo+ phagosome containing E. Coli bioparticles in ACLF and healthy blood monocytes on at least 40 cells from 15 fields/individual. 63× Magnification, scale bar: 5 μm. Results from cirrhotic patients cirrhosis (dashed lines) were included for comparison. (F). Quantification of LC3+ pHrodo+ phagosome containing E. Coli bioparticles from ACLF blood monocytes (n=7) exposed to either vehicle, antibodies to FcγRIIA F(ab′)2 or 10 mg/ml IVIg for 3 hrs. 63× magnification, scale bar: 5 μm. *p<0.05 for control vs FcγRIIA F(ab′)₂ antibody or IVIg. Statistical analysis by Mann-Whitney (a, b, c, d, e) or Wilcoxon matched-pairs signed rank (f) tests.

### EXAMPLE

Methods:

Human blood samples. Following approval by local Ethics committee (Comité de protection des personnes Ile de France III N^{o} 3194 and Comité d'Evaluation de l'Ethique des projets de Recherche Biomédicale (CERB) Paris Nord (IRB 00006477 n^{o} 13-043)) and written informed consent, blood samples were obtained from 64 patients with histologically-proven cirrhosis admitted to liver unit (n=64 with stable cirrhosis) or to intensive care unit (n=21 patients with acute on chronic liver failure) from Beaujon university hospital. Non-inclusion criteria of patients with stable cirrhosis were: i) an acute event (hepatorenal syndrome, bacterial infection, variceal bleeding) within two weeks before inclusion, ii) Current treatment with immunosuppressive drugs, iii) Current active alcohol consumption, iv) Presence of human immunodeficiency virus infection and ivv) hepatocellular carcinoma (HCC) outside Milan criteria or active extrahepatic cancer. In patients with ACLF (defined according to⁶), extensive bacteriological exams were performed to search for bacterial infection as precipitating event. Blood from healthy volunteers (n=70) was obtained by Etablissement Français du Sang (agreement no 2015012778).

Human Liver samples. Liver samples were obtained from surgical samples (resection or liver transplantation). In cirrhotic patients, samples were obtained from liver explant during liver transplantation or from non-tumoral liver during hepatocellular carcinoma resection. Control liver samples were taken from patients with normal liver biological tests who underwent resection surgeries for non-hepatocellular primary tumor or colorectal cancer liver metastasis. Every liver specimen was analyzed by a pathologist expert for liver diseases. All patients signed an informed consent form and the study was approved by the local Ethics Committee.

Mice. Human FcγRIIA-Tg mice expressing the human FcγRIIA^{R131} in CD11b+ cells were from Jackson Laboratory (JAX, Bar Harbor, Me., USA). All mice were of C57BL/6 strain and mice carrying the FcγRIIA transgene were used as heterozygous animals. WT littermates were used as controls. Myeloid cell-specific Rubicon-deficient mice were generated by crossing RBCN-loxP/loxP (²², kindly provided by Dr T Yoshimori (Osaka University, Japan) to LysM-Cre mice (Jackson Laboratory, Charles River France, L'Arbresle, France), and backcrossing the resulting double heterozygotes (LysM-Cre+/−, RBCN+/loxP) with RBCN-loxP/loxP mice to produce myeloid-specific Rubicon knockout mice (LysM-Cre+/−, RBCN-loxP/loxP, Rubicon^{Mye-/-} and wild-type littermates (LysM-Cre−/−, RBCN-loxP/loxP, Rubicon^{flox/flox} mice). Rubicon^{flox/flox}LysMCre^{-/-} littermates were used as controls.

Mice models of severe liver fibrosis. Animals were housed in pathogen-free animal facility and fed ad libitum. Liver fibrosis was induced in male mice (8-11 week-old) by either oral gavage of carbon tetrachloride (CCl₄, 0.6 ml/kg body weight, Sigma-Aldrich, 2700652), 1:10 dilution in mineral oil (MO, Sigma-Aldrich, M5310), twice a week for 7 or 10 weeks, or bile duct ligation. Forty eight hours after the last gavage, or 13 days after surgery, mice were administered one single dose of LPS (2 mg/kg, Sigma, L3024-5MG, Lot: 115M4223V) by intraperitoneal injection and sacrificed after 24 hrs, as described in^{40,41}. Experiments were performed in accordance with protocols approved by in accordance with the French Council of Animal Care guidelines and national ethical guidelines of INSERM Animal Care Committee (authorization number 02529.02).

Histological analysis. Picrosirius red staining and hematoxylin and eosin (H.E.) staining were performed on 4 μm thick formalin-fixed paraffin-embedded mice liver tissues at the Pathology department of Hôpital Bichat, Paris. Images were taken at 10× magnification and morphometric pixel analysis of or infiltrated area on 10 non-overlapping randomly chosen fields per mouse, using ImageJ software (NIH, USA). Images taken at 20× magnification were shown as representative image.

Immunohistochemistry. Immunohistochemical detection of α-Smooth Muscle Actin (α-SMA) was carried out as previously described⁴² on formalin-fixed paraffin-embedded mice liver tissue sections (4 μm) using the MOM immunodetection kit (Vector Laboratories, BMK 2202) and a mouse monoclonal anti-α-SMA antibody (1:1000, Sigma-Aldrich, A2547) according to manufacturer's instructions. α-SMA positive areas from 10 fields (magnification ×10)/mouse liver were quantified using ImageJ software (NIH, USA). No staining was observed when omitting the primary antibody.

Peripheral blood mononuclear cells (PBMC) and monocyte isolation. PBMC were prepared from the blood of cirrhotic patients and healthy donors, using Ficoll density gradient centrifugation (Ficoll Paque™-plus, GE Healthcare, 17-1440-02), as previously described⁴³.

Human blood monocytes. Human monocytes were isolated from freshly isolated PBMC, using EasySep human monocyte enrichment kit (STEMCELL Technologies, 19058), and CD14+CD16+ monocytes were isolated by immunomagnetic negative selection according to the manufacturer's instructions.

Human and mouse intrahepatic monocytes. Human intrahepatic leucocytes were isolated following mechanical dissociation of fresh human liver pieces (2 g tissue), and Ficoll density-gradient centrifugation at 800 g, as previously described⁴³. Mouse intrahepatic leukocytes were isolated by digestion with Liberase™ (Sigma-Aldrich, 5401119001) and Ficoll density-gradient centrifugation, as described in⁴³. Intrahepatic monocytes were then isolated from leukocytes by adherence at 37° C. for 2 hrs in RPMI containing 10% FCS.

Bone-marrow derived and peritoneal macrophages were isolated as previously described³⁴

Cell treatment. Human monocytes, THP1-XBlue™-CD14 (Invivogen, Catalog #thpx-cd14sp) and THP1-FcγRIIA+−CD14+(kindly provided by Novimmune) monocytes were used. When indicated cells were exposed to either 30 μM chloroquine diphosphate (Sigma, C6628), 10 mM 3-Methyladenine (3-MA, Sigma, M9281-100 mg), 10 mM Diphenyleneiodonium chloride (DPI, Sigma, D2926-10 mg), or 10 mg/ml IVIg (Privigen, 100 mg/ml) for 4 hrs at 37° C., as indicated. We also used commercially synthetized Tat-conjugated peptides (30 μM, GeneCust, Dudelange): i) the Tat-p22phox N8 peptide corresponding to eight N-terminal amino acids (3-10) of p22phox that blocks the Rubicon-p22phox interaction¹⁹; ii) its control mutant Tat-N8W9A peptide, in which one tryptophan residue is replaced by Alanine¹⁹). In some experiments, cells were exposed to the preformed complex (10 μg/ml of mouse anti-hFcγRII mAb (clone AT-10, Santa Cruz, sc-13527) F(ab′)₂ fragments+20 μg/ml goat anti-mouse anti-kappa F(ab′)₂ fragments (Southern Biotech, 1052-01) or to 100 μg/ml of anti-hFcγRIIA mAb F(ab′)₂ fragments (clone IV.3), purified as we previously described²⁶.

HPLC fractionation of serum samples and IgG detection. Serum samples (500 μl) from cirrhotic patients, healthy donors and patient with lupus nephritis were diluted in PBS and resolved by gel filtration through a Superdex 200 10/30 column (GE Healthcare, 10/300GL 17-51-75-01) connected to an HPLC AKTA-basic automated liquid chromatography system (GE Healthcare). Sera or HPLC fractions were solubilized in SDS sample buffer under non-reducing conditions and subjected to electrophoresis in 4-15% polyacrylamide gel. Proteins were electroblotted on polyvinylidene difluoride membranes (Millipore) and subjected to Western blot analysis using a biotinylated goat anti-human IgG coupled to horseradish peroxidase (HRP) (1/40000, Bethyl laboratory, A80-104P).

Immunoprecipitation and immunoblotting. Human serum fractions, human PBMCs, human monocytes, mouse bone marrow-derived macrophages (10⁶ to 10⁷) were solubilized in RIPA lysis buffer containing 1% Nonidet P-40/0.1% sodium-dodecyl-sulfate (SDS) as we described⁴². For immunoprecipitation, human monocyte lysates were incubated with 2 μg/ml of AT-10 anti-hFcγRII mAb (sc-13527, Santa Cruz) F(ab)₂ fragment and immunoprecipitated overnight at 4° C. with Protein G-Sepharose (GE Healthcare). Immunoblotting was performed with either rabbit anti-LC3B antibodies (1:500, Novus Biologicals, Biotechne, NB100-2220), Rabbit anti-phospho AMPKα (Thr172) (1:500, 40H9, Cell signaling, 2535), guinea pig anti-p62 (1:1000, Progen Biotechnik, GP62-C), rabbit anti-ULK1 (1:1000, D8H5, cell signaling, 8054), rabbit anti-phospho ULK1 ser757 (1: 1000, cell signaling, 6888), rabbit anti-Rubicon antibody (1:1000, cell signaling, 8465), mouse anti-SHP-1 (SH-PTP1 (D-11), Santa Cruz, sc-7289) and mouse anti-β actin (1:10000, Sigma-Aldrich, clone AC, A5441-5ML,) followed by horseradish peroxidase (HRP)-conjugated anti-IgG antibodies, either goat anti-rabbit (1:2,500, Jackson ImmunoResearch, 711-035-152) or goat anti-mouse (1:10,000, GE healthcare, NA9310), donkey-anti-mouse (1:10000, Jackson ImmunoResearch, 715-005-151) or donkey-anti-guinea pig (1:2500, Jackson ImmunoResearch, 706-035-148). Membranes were developed by Amersham ECL prime western blot detection reagents (Amersham Biosciences, RPN2236).

siRNA transfections. Experiments were performed using predesigned HP GenomeWide (Qiagen, Courtaboeuf, France) siRNAs. Single-strand sense and anti-sense RNA nucleotides were annealed to generate an RNA duplex according to the manufacturer's instructions. Cells were incubated with 5-10 nM siRNA and 2 μl of Lipofectamine RNAiMAX prepared according to the manufacturer's instructions (Invitrogen, Saint Aubin, France) for 48 or 72 hrs at 37° C. before use.

Flow cytometry. For surface and intracellular staining, PBMC (1×10⁶) were centrifuged at 4° C. at 500 g for 5 min and stained in the dark for 30 min at 4° C. in flow cytometry staining buffer (5% FCS-PBS) containing fluorochrome-conjugated antibodies (APC-CD14, clone HCD14, Biolegend, 325608), CD11b-PE-vio 770 (clone REA592, Miltenyi Biotec, 130-109-287), CD16 Brilliant violet 605 (clone3G8, 302040, Biolegend) and FITC-FcγRIIA (clone IV.3, 1:100, Stemcells Technologies, 60012FI). For intracellular staining, cells were washed, permeabilized in Perm-wash buffer (BD Biosciences, 554723) and incubated in the dark for 30 min at 4° C. with either PerCP anti-human IL8 (clone BH0814, Biolegend, 514606), FITC-IL6 (clone MQ2-13A5, Biolegend, 501104) or with anti-FITC-FcγRIIA, and filtered at 40 μM cell strainer before acquisition. LIVE/DEAD Fixable viability dye eFlour 506 (Invitrogen, 65-0866-14) was used to discriminate between live and dead cells. Data acquisition was performed using a BD Biosciences LSR Fortessa cytometer and data were analysed using FlowJo analysis software (Tree Star).

In vitro phagocytosis assays. pHrodo™ Red IgG latex beads were prepared as described in⁴⁴ from Fluoresbrite® BB Carboxylate Microspheres 1.79 μm (2% solids-Latex) (Polysciences Inc. (17686, Warrington, Pa., USA) conjugated with chromPure human IgG (009-000-003, ImmunoResearch Lab). In vitro phagocytosis was performed as previously described⁴⁵. Briefly, 0.1×10⁶ cells were seeded onto fibronectin-coated glass coverslips in 24 well plate. pHrodo™ red E. coli Bioparticles (ThermoFisher scientific, A10025) or IgG latex beads were added to the cells at a ratio of 6.25:1 (bioparticles: cells) for 15 min at 37° C., and cells were fixed with 4% PFA (Sigma, P6148-1KG).

Immunofluorescence. Following incubation in PBS containing 0.2% BSA and 0.2% saponin, fixed cells were incubated for 1 hr at room temperature with either rabbit-anti LC3 antibody (1:200, Novus Biologicals, Biotechne, NB100-2220), mouse anti-human p22phox (1:200, Santa Cruz, sc-130550), rabbit anti-PI3K class III (1:200, Cell Signaling, 4263), mouse anti-human IgG (1:100, Jackson ImmunoResearch 209-005-082), rabbit anti-PTPN6 (TYR536) conjugated Alexa Fluor 488 (1:100, Bioss, bs-5577R-A488) or rat LAMP1 (1: 200, clone 1D48, Abcam, ab25245). Slides were then incubated for 30 min with secondary antibody, either goat anti-rabbit Alexa488 (1:100 or 1000, ThermoFisher scientific, A11034), goat anti-mouse Alexa488 (1:100, ThermoFisher scientific, A11001), goat anti-mouse Alexa647 (1:100, ThermoFisher scientific, A21236) or goat anti-rat (1:100, ThermoFisher scientific, A21434). Cells were mounted by ProLong™ Gold Antifade Mountant with DAPI (ThermoFisher scientific, P36935). Representative images were taken using Confocal SP8 Leica microscope, equiped with a 63× oil immersion objective. For quantification, images were acquired by confocal microscopy (Confocal Zeiss LSM 780) equipped with a 63× oil immersion objective. For each condition, 10 to 15 fields were randomly selected, and the number of LC3+ signal localized around pHrodo+ phagosomes (LC3+ pHrodo+) was quantified in at least 40 to 60 cells. Unless otherwise indicated, results are expressed as number of LC3+ phagosomes/individual normalized to 100 cells. No staining signal was observed when omitting the primary antibody. Specificity of mouse anti-human IgG labelling was also assessed using irrelevant rabbit IgG, which showed no signal.

Quantification of cytokines. IL1-β and IL6 were quantified in 200 mg of mouse liver lysates using ELISA kits according to the manufacturer's instructions (88-7013-88 and 88-7064-88).

RNA preparation and Real-time PCR. Total RNA was extracted from 1×10⁶ human monocytes or 20 mg of mouse liver fragments from the median and left lobes by using 1 ml Qiazol Lysis reagent (Qiagen, 79306), and RNAeasy mini columns (Qiagen, 74104), as previously described⁴². Reverse transcription was performed on 1 μg RNA using either Verso cDNA synthesis kit (Thermo Fisher Scientific, AB-1453/B) or High Capacity cDNA reverse transcriptase kit (Thermo Fisher Scientific, 4368813) for human and mouse RNA, respectively. Real time-PCR was performed with ABsolute Blue QPCR SYBR Green Low ROX Mix (Thermo Fisher Scientific, AB-4166/B). Gene expression was normalized to GAPDH or 18S rRNA for human and mouse genes, respectively.

Microarray. The expression of LAP receptor genes was analysed from microarray expression data, using an Affymetrix Human Exon Array in PBMC from 4 patients with cirrhosis and 4 healthy subjects that we described in²³.

Liver function. Serum levels of alanine and aspartate aminotransferase were measured at the Plateforme de Biochimie, INSERM U 1149, Paris, France.

Statistical analysis. Results are expressed as mean±standard error of the mean (SEM) or median (interquartile range (IQR)), as indicated. Comparison between groups were performed using appropriate non parametric tests, Mann-Whitney or Wilcoxon matched-pairs signed rank tests for continuous variables and chi-square test for categorical variables. Correlations were performed using non-parametric Spearman test. All p values are 2-sided, and p values less than 0.05 were considered to be statistically significant. The potential relationship between patient characteristics and LAPosome number was analysed by linear regression univariate analysis. Each variable achieving a p value<0.05 was then introduced into a bivariate model. Analyses were performed using GraphPad Prism version 8 and SPSS 22.0 (SPSS Inc. Chicago, Ill., USA).

Results:

LAP Protects Against Systemic and Hepatic Inflammation, with Antifibrogenic Effects in the Liver.

We performed studies in healthy donors and in a cohort of patients with cirrhosis from different etiologies (alcoholic or non-alcoholic fatty liver disease and Viral Hepatitis C. We first evaluated the status of LC3-II, a marker for autophagy, and LAP in peripheral-blood mononuclear cells (PBMC) and monocytes from patients with cirrhosis, as compared to healthy donors. Western blot analysis of protein extracts from PBMC showed a significant increase in LC3-II lipidation in cirrhotic patients, as compared to healthy donors, that was observed whatever the etiology (data not shown). In addition, monocytes from cirrhotic patients showed higher endogenous LC3 fluorescence intensity, as compared to healthy counterparts (data not shown). Increased LC3-II lipidation was further enhanced by the lysosomal pH inhibitor chloroquine in PBMC from cirrhotic patients, and affected more marginally cells from healthy donors (data not shown).

Because both LAP and autophagy control inflammation in monocytes, we investigated whether enhanced LC3-II lipidation in patients with cirrhosis is related to LAP or autophagy. We investigated the recruitment of LC3 to phagosomes in blood monocytes from cirrhotic and healthy subjects, following uptake of either IgG-coated beads (data not shown) or pHrodo-Red E. coli BioParticles™ (data not shown). Recruitment of LC3 to pHrodo+ phagosomes (LAPosome) was minimal in monocytes from healthy donors, but strongly increased in cells from patients (data not shown). This association between cirrhosis and monocyte LAPosome number was confirmed using linear regression (data not shown). It should be noted that although gender was another parameter associated with laposome number, bivariate analysis adjusted on gender showed that cirrhosis was still an independent predictor of higher LAPosome number (data not shown).

Recruitment of LC3 to pHrodo+ phagosomes paralleled that of PI3KIII Vps34 and of the p22phox subunit of the NADPH complex (data not shown). Concordantly, disrupting LAP components by either the PI3KCIII inhibitor 3-Methyladenine (3-MA)¹⁸ or with an N-terminal 8-amino acid peptide derived from p22phox coupled to HIV-Tat protein (N8 peptide), which uncouples p22phox-Rubicon interaction¹⁹, led to a significant reduction in LC3+ phagosome number in monocytes from patients (data not shown). In contrast, the level of the autophagy cargo protein p62²⁰ did not vary between PBMC from both groups (data not shown). Moreover, activation of the early macroautophagic steps was not observed in PBMC from patients with cirrhosis. In particular, there was no modification of Phospho-AMPKα (Thr172) as compared to healthy donors (data not shown), but cells from patients showed enhanced phosphorylation of ULK1 on Ser⁷⁵⁷, a phosphorylation site that prevents ULK1 activation and its interaction with AMPK²¹, (data not shown). These data suggested that LC3-II lipidation is mostly related to LAP rather than canonical autophagy in circulating monocytes from patients with cirrhosis.

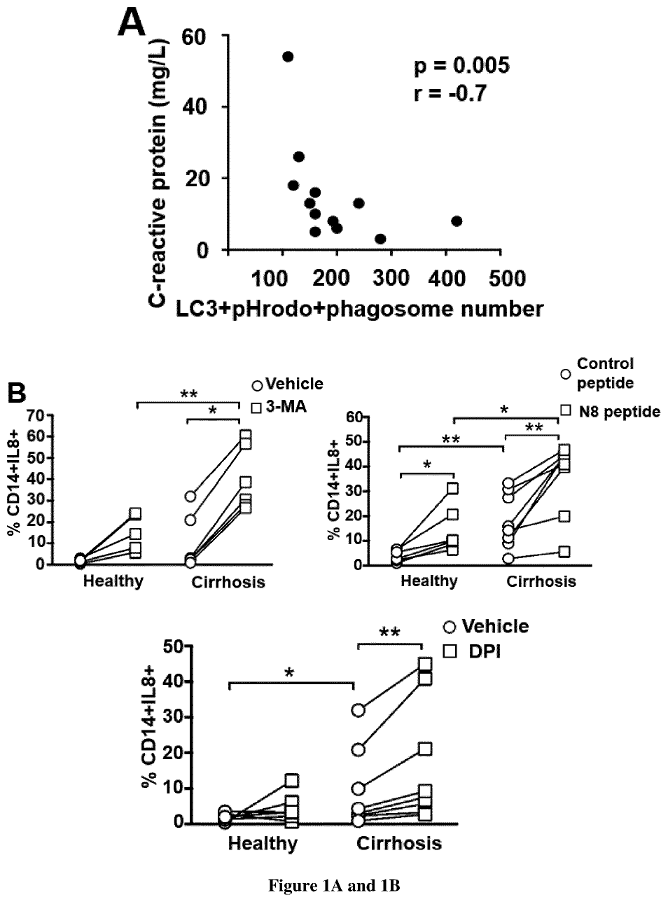

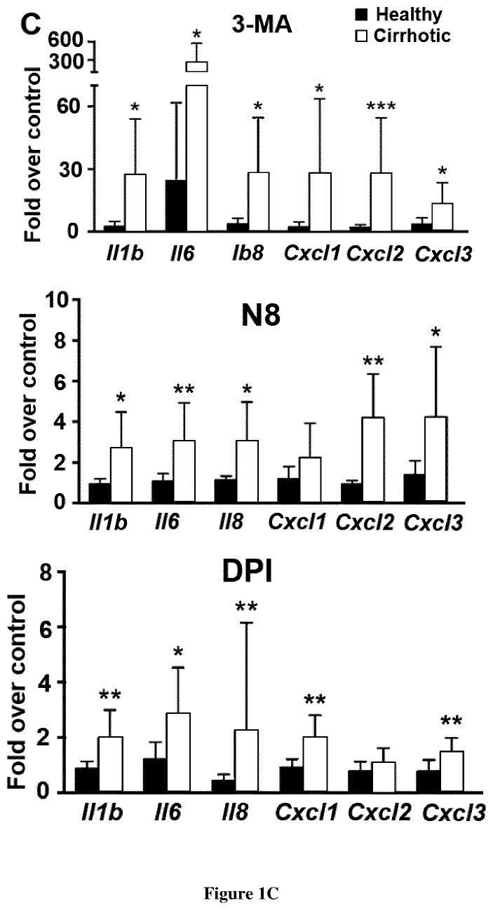

We therefore focused on LAP and further investigated the link between LAP activation and inflammation in monocytes from patients with cirrhosis. Interestingly, we found an inverse correlation between the number of LAPosomes and blood levels of C-Reactive Protein, an inflammatory biomarker routinely used in clinics (FIG. 1A). Moreover, both 3-MA and N8 peptide enhanced the inflammatory signature in blood monocytes from both healthy donors and patients, with a prominent increase in cells from patients as compared to that of healthy donors (FIG. 1B and 1C). In particular, the number of CD14+IL8+ monocytes (FIG. 1B), and parallel gene expression of cytokines and chemokines (FIG. 1C) were significantly higher in cirrhotic monocytes exposed to 3-MA or N8 peptide, as compared to control-treated cells from healthy donors. Similar findings were obtained when using the NOX inhibitor DPI (FIG. 1B and 1C). Taken together, these data indicated that LAP is activated in blood monocytes from patients with cirrhosis and restrains systemic inflammation.

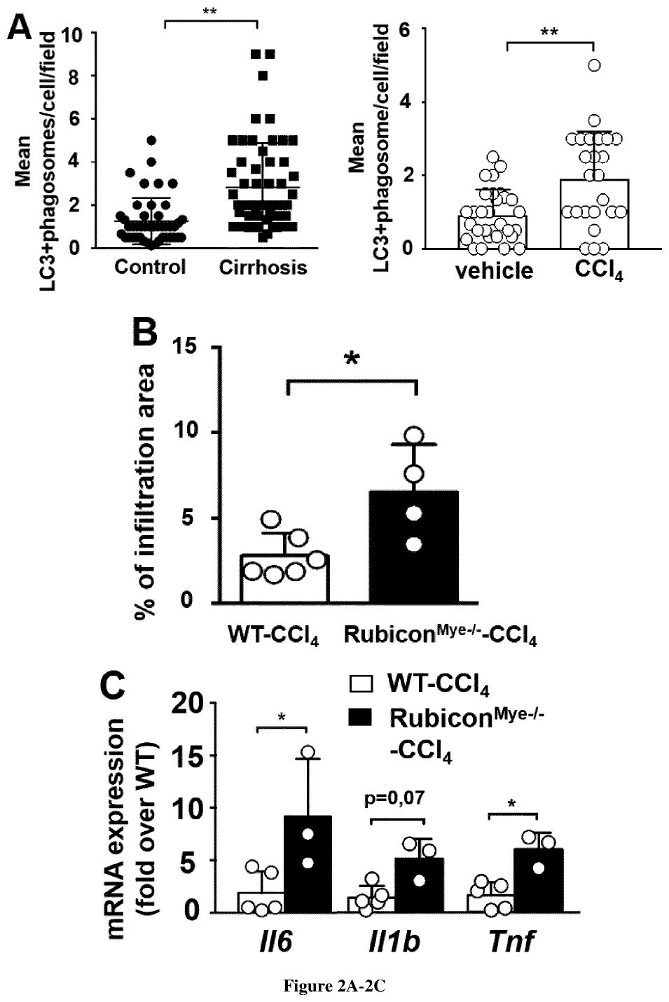

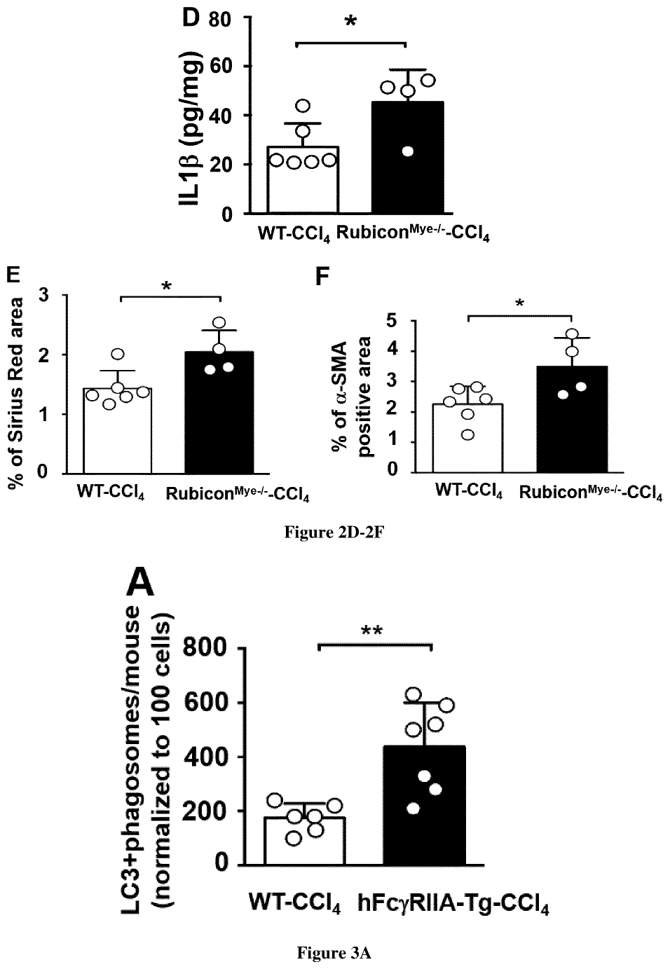

Recruitment of LC3 to pHrodo+ phagosomes was also enhanced in monocytes/macrophages isolated from cirrhotic livers, as compared to cells isolated from control livers (FIG. 2A). Increase in LAPosome number was also observed in intrahepatic monocytes/macrophages isolated from mice with liver fibrosis, induced by either chronic administration of CCl₄ (FIG. 2A) or bile duct ligation (BDL) (data not shown). We investigated the consequences of LAP blockade on inflammation and chronic liver injury, and developed mice lacking Rubicon in the myeloid lineage (Rubicon^{Mye-/-}) by crossing Rubicon^{flox/flox 22} with transgenic mice expressing the recombinant Lysozyme-M Cre recombinase. The efficiency of the deletion was confirmed by the decreased Rubicon protein expression in bone marrow-derived macrophages (BMDM) from Rubicon^{Mye-/-} mice (data not shown). In addition, the number of LAPosomes in BMM from CCl₄-exposed Rubicon^{Mye-/-} mice was similar to that of vehicle counterparts, and strongly decreased as compared to that of CCl₄-exposed Rubicon^{flox/flox} mice (data not shown). Deletion of Rubicon in myeloid cells had no effect on CCl₄- or BDL induced liver injury, as reflected by similar levels of serum alanine transaminase (ALT) in Rubicon^{Mye-/-} mice and WT counterparts (data not shown). However, CCl₄-exposed Rubicon^{Mye-/-} mice showed enhanced inflammatory infiltrate in the liver (FIG. 2B), associated with enhanced hepatic inflammatory cytokine expression and secretion (FIG. 2C-2D). Moreover, CCl₄-exposed Rubicon^{Mye-/-} mice displayed exacerbated fibrosis, as evidenced by enhanced Sirius red staining area (FIG. 2E) and increased number of fibrogenic cells, evaluated by α-smooth muscle actin (α-SMA) staining (FIG. 2F). Similar results were obtained in Rubicon^{Mye-/-} BDL mice (data not shown). These data demonstrate that in response to chronic liver injury, mice deficient in LAP in myeloid cells develop exaggerated hepatic inflammation and are more prone to develop liver fibrosis.

IgG-Mediated Activation of the Immunoreceptor FcγRIIA Underlies LAP-Mediated Antiinflammatory Signaling

We next characterized the mechanisms underlying endogenous activation of LAP in cirrhotic monocytes and first evaluated the expression of several plasma membrane LAP-triggering receptors, using available microarray results comparing baseline PBMC transcriptome between healthy donors and patients with cirrhosis²³. We found that gene expression of the Fcγ immunoreceptor FCGR2A was significantly higher in patients' PBMC as compared with donors, whereas neither the expression of CLEC7A nor that of the antiinflammatory FCGR2B differed between the groups, and that of TIM4 was even slightly decreased (data not shown). FACS analysis confirmed that the expression of FcγRIIA in cirrhotic monocytes was increased both at the cell surface and intracellularly (data not shown). FcγRIIA is a low affinity IgG receptor that binds all human and mice IgG²⁴. Interestingly, it is known for decades that patients with chronic liver diseases show hyperimmunoglobulinemia²⁵, but the consequences on the inflammatory features of monocytes during cirrhosis remain unclear. Strikingly, a strong increase in IgG immunostaining was observed in monocytes from patients with cirrhosis compared to healthy donors (data not shown). IgG immunostaining was mainly colocalized in LC3+LAMP1+ compartments in monocytes from patients, but not in monocytes from healthy donors (FIG. 4b). LAMP1 immunostaining, commonly used as a phagosome maturation marker, increased in parallel to that of LC3 in patient monocytes, suggesting that, as previously reported, LC3 recruitment allows phagosome maturation^{9,10} These data indicate that during cirrhosis, IgG engulfed into phagosomal structures in monocytes lead to the recruitment of the LAP machinery and to the initiation of an antiinflammatory response.

FcγRIIA can function as a bi-functional receptor to trigger either pro- or anti-inflammatory signals depending on the type of ligand, a property that can be exploited to modulate inflammatory disease development^{26,27}. Thus, crosslinking of FcγRIIA by multimeric IgG immune complexes results in the phosphorylation of Immunoreceptor Tyrosine-based Activation Motif (ITAMa) tyrosine residues, triggering pro-inflammatory signals²⁸⁻³⁰. In contrast, upon interaction with uncomplexed IgG (either in monomeric or dimeric forms) or with specific antibody F(ab′)₂ fragments, the receptor generates inhibitory ITAM (ITAMi)-dependent signals³¹ with anti-inflammatory effects, following stable recruitment and activation of the phosphatase SHP-1^{26,27}. In order to determine whether serum IgG from cirrhotic patients were under complexed or uncomplexed form, we analyzed serum IgG fractions from size chromatography column by SDS-PAGE on non-reducing conditions. We found that serum IgG from cirrhotic patients were essentially in monomeric forms with small amounts of dimers, but did not contain high molecular weight IgG complexes, whereas as previously reported³², serum IgG from a patient with lupus nephritis displayed high molecular weight IgG complexes (data not shown). Moreover, cirrhotic patients showed increased amounts of both monomeric and dimeric IgG, as compared to healthy donors (data not shown). We therefore hypothesized that interaction of uncomplexed IgG with FcγRIIA may drive LAP to inhibit inflammation via ITAMi in cirrhosis. We thus first explored the consequences of either multivalent cross-linking or mono/divalent targeting of the receptor in patient monocytes, thereby mimicking interactions with complexed or uncomplexed forms of IgG, respectively. The induction of ITAMa, that blunts ITAMi signaling^{26,27} promoted by crosslinking of FcγRIIA with a preformed complex (anti FcγRII F(ab′)₂-fragments+anti-kappa chain) decreased the number of LC3+ phagosomes in patient monocytes (data not shown). This effect was associated with an increased capacity of patient monocytes to produce proinflammatory cytokines and chemokines (data not shown). In contrast, stimulation of ITAMi signaling upon exposure to IVIg of THP-1 monocytes transfected with the human FcγRIIA, resulted both in an increase in the number of LC3+ phagosomes (data not shown) and of LC3-II lipidation (data not shown), as compared to untreated cells. Furthermore, immunoprecipitation of FcγRIIA revealed its association with SHP-1 in IVIg-stimulated THP-1 cells, but also its interaction with LC3 (data not shown). Interestingly, association of FcγRIIA/SHP-1 with LC3 was also observed in FcγRIIA immunoprecipitates from monocytes of patients with cirrhosis but not of healthy donors (FIG. 4f). Moreover, the activated phosphorylation form of SHP-1 (p-SHP-1^{Y536}) colocalized with pHrodo+ phagosomes in patient monocytes (data not shown). In keeping with these results, siRNA silencing of SHP-1 blocked both the increase in LC3 expression and its interaction with FcγRIIA in immunoprecipitates from THP-1 monocytes transfected with the human FcγRIIA, as compared to control siRNA (data not shown). Altogether, these data demonstrate that during cirrhosis, engulfment of IgG by monocytes promotes FcγRIIA-mediated ITAMi signaling, which recruits LC3 and promotes LAP.

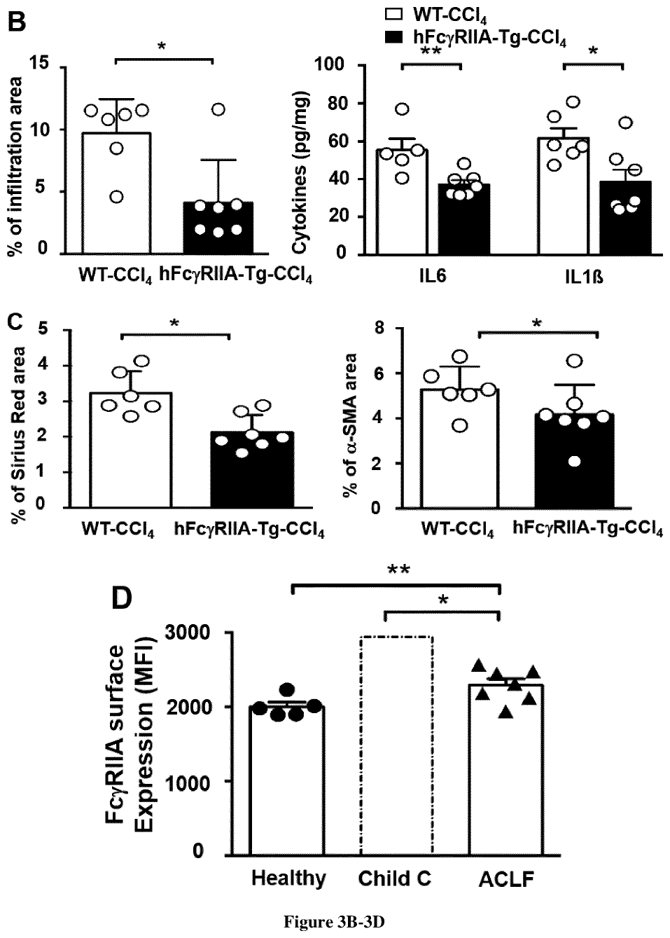

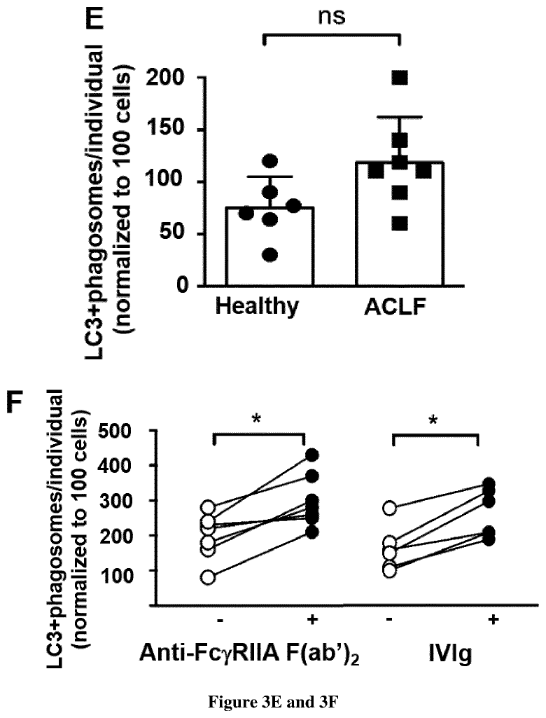

We next examined the consequences of human FcγRIIA overexpression in a mice model of advanced chronic liver disease, following 10 week administration of CCl₄, using animals overexpressing human FcγRIIA in myeloid cells (hFcγRIIA-Tg mice) and their WT counterparts. When exposed to CCl₄, hFcγRIIA-Tg mice showed an increase in the number of LAPosomes, as compared to WT counterparts (FIG. 3A and data not shown). Moreover, whereas CCl₄-exposed WT mice displayed massive hepatic inflammatory infiltrate and severe liver fibrosis (FIGS. 3B and 3C), CCl₄-exposed hFcγRIIA-Tg animals were more protected and showed reduced inflammatory infiltrate in the liver and lower production of hepatic inflammatory cytokines (FIG. 3B). They were also more resistant to liver fibrosis than WT counterparts, as shown by lower Sirius red staining and reduced number of α-SMA positive cells (FIG. 3C).

Patients with cirrhosis are prone to develop ACLF, and the severity of organ failure and mortality rate in ACLF patients is linked to the intensity of systemic inflammation⁴⁻⁶. We therefore hypothesized that FcγRIIA-mediated activation of LAP observed in circulating monocytes from patients with cirrhosis may be switched off in those with ACLF. As compared to blood monocytes from patients with cirrhosis, the increase in FcγRIIA expression was much lower in ACLF patients (FIG. 3D and data not shown). In addition, there was no increase in the number of LAPosomes in monocytes from patients with ACLF, as compared to healthy donors (FIG. 3E and data not shown). Interestingly, incubation of ACLF monocytes with anti-FcγRIIA F(ab′)₂ fragments was able to increase the number of LAPosomes (FIG. 3F). Moreover, restoration of LAP was also observed upon exposure of ACLF monocytes to IVIg, which are already used for their immunoregulatory properties in the treatment of some inflammatory diseases³³.

Discussion:

In the present study, combining studies in human samples and mice models, we identify LC3-associated phagocytosis as an anti-inflammatory pathway in monocytes, that constrains both systemic and hepatic inflammation during cirrhosis, with potent antifibrogenic effects. Importantly, this mechanism is lost when cirrhosis deteriorates to acute on chronic liver failure, a severe form of the disease characterized by a burst in systemic inflammation and a high risk of mortality⁴⁻⁶, but can be restored upon exposure of monocytes to monomeric IgG.

Our data support a role for LAP, rather than canonical autophagy, in the anti-inflammatory signal carried by monocytes from patients with cirrhosis, based on the lack of variations in AMPK phosphorylation, the increase in the inhibitory phosphorylation of ULK1 and the absence of p62 modulation. In contrast, enhanced recruitment of the LAP machinery proteins is observed in LC3-decorated phagosomes, including Vps34 and p22phox. Concordantly, blockade of LAP by blunting either Vps34, NADPH oxidase or Rubicon-p22phox interaction exacerbates the inflammatory signature of cirrhotic blood monocytes, suggesting that LAP prevents reprogramming of monocyte/macrophage to a proinflammatory phenotype in cirrhotic patients. The role of LAP is further corroborated by studies in the liver, showing that this pathway is also increased in hepatic monocytes upon chronic liver injury in cirrhotic patients. Moreover, mice deficient for LAP in myeloid cells show pronounced hepatic inflammatory cell infiltration and cytokine production in response to chronic liver injury, a step required for the development and progression of liver fibrosis to more severe stages^{1-3,17,34} Accordingly, LAP-deficient mice in myeloid cells are more prone to develop fibrosis in response to chronic liver injury, therefore highlighting a novel role for LAP in the regulation of inflammation-driven fibrogenesis. These data are in line with our recent results showing a similar phenotype in mice bearing deletion of ATG5 in myeloid cells, that develop exaggerated hepatic inflammation and fibrosis when chronically exposed to CCl₄¹⁷. However, because autophagy and LAP share similar and overlapping sets of proteins, a role for both pathways in the regulation of inflammation during cirrhosis cannot be entirely ruled out.

Several signals elevated during cirrhosis^{1,2} could mediate LAP activation, including apoptotic cells via TIM-4, PAMPs via Pattern-Recognition Receptors, or immunoglobulins via Fcγ receptors. Interestingly, hyperimmunoglobulinemia has been reported as a characteristic feature of cirrhosis²⁵. Importantly, our data further demonstrate that the serum of patients with cirrhosis show increased levels of uncomplexed monomeric/dimeric IgG, without high molecular weight complexes, a characteristic feature of autoimmune diseases such as lupus nephritis³². Moreover, we found enhanced FCGR2A expression, but not TIM4 or CLEC7A expressions in blood monocytes from patients with cirrhosis, that was associated with an increase in IgG particle engulfment, suggesting that IgG-FcγRIIA signaling is a likely candidate for LAP activation. FcγRIIA has double edge sword functions, with anti- and pro-inflammatory properties depending on specific core signaling pathways^{26,27}. Interestingly, our results also unravel a beneficial role for FcγRIIA during chronic liver injury, since mice overexpressing FcγRIIA in myeloid cells are protected against hepatic inflammation with a resulting resistance to liver fibrosis, when exposed to chronic toxic insult. Mechanistically, we have previously described that the inhibitory signal transmitted by FcγRIIA is generated via recruitment of SHP-1 to the receptor, following its activation by monomeric IgG^{26,27} Strikingly immunoprecipitation studies of monocyte lysates from patients with an anti FcγRIIA antibody revealed association of the receptor with LC3, an interaction that was blunted upon SHP-1 silencing. These data provide a novel unsuspected positive link between LAP and FcγRIIA-SHP-1-ITAMi signaling to convey an antiinflammatory response. Previous studies have shown that immune receptors associated with an ITAM motif, such as Dectin-1, are able to enhance LAP following recruitment of LC3 to the phagosome via the kinase Syk^{35,36} This kinase is required for initiation of signaling by receptors that utilize ITAM domains, and is negatively regulated by SHP-1^{26,27}. However, we have also previously shown that transient activation of Syk by monomeric IgG is needed for recruiting SHP-1 by FcγRIIA and transmitting an anti-inflammatory signal^{26,27}. In addition, our data are in line with previous reports showing that SHP-1 associates with phagosomes and promotes their biogenesis³⁷. Therefore, whether Dectin-1 promotes LAP via an ITAMi-SHP-1-dependent pathway merits further investigation, especially in the context of chronic liver diseases, in which Dectin-1 displays anti-inflammatory and antifibrogenic properties³⁸. Furthermore, it is tempting to hypothesize that the antiinflammatory effects promoted by FcγRIIA-ITAMi signaling in experimental arthritis or lupus nephritis^{26,27} involves LAP. Finally, the beneficial role of LAP in other human inflammatory disorders also merits further investigation, in light of our results in cirrhosis, and those reported in mice for lupus erythematosus³⁹.

Our data broaden our knowledge on the general properties of LC3-associated phagocytosis in the regulation of inflammation, and our understanding of the mechanisms underlying inflammation-driven fibrogenesis and systemic inflammation in the context of cirrhosis. From a clinical perspective, our study also has potential major implications. Progression of cirrhosis to acute on chronic liver failure, the most severe form of cirrhosis, is a consequence of an acute accentuation of systemic inflammation over the chronic systemic inflammation already present in cirrhosis⁴⁻⁶. However, the mechanisms underlying the burst in inflammation are not fully understood. Our data demonstrate that activation of LAP observed in blood monocytes from patients with cirrhosis is lost in patients progressing to ACLF. Most interestingly, LAP can be restored upon exposure of monocytes from ACLF patients to the already FDA-approved IVIg approach, or by specifically targeting FcγRIIA by antibodies to FcγRIIA F(ab′)₂ fragments. These data suggest that sustaining LAP in cirrhotic patients could prevent progression to a syndrome which is associated with a high mortality rate.

### REFERENCES

Throughout this application, various references describe the state of the art to which this invention pertains. The disclosures of these references are hereby incorporated by reference into the present disclosure.
1. Mallat, A. & Lotersztajn, S. Cellular Mechanisms of Tissue Fibrosis. 5. Novel insights into liver fibrosis. Am J Physiol Cell Physiol 305, C789-799 (2013).
2. Lotersztajn, S., Julien, B., Teixeira-Clerc, F., Grenard, P. & Mallat, A. Hepatic fibrosis: molecular mechanisms and drug targets. Ann Rev Pharmacol Toxicol 45, 605-628 (2005).
3. Krenkel, O. & Tacke, F. Liver macrophages in tissue homeostasis and disease. Nat Rev Immunol 17, 306-321 (2017).
4. Claria, J., Arroyo, V. & Moreau, R. The Acute-on-Chronic Liver Failure Syndrome, or When the Innate Immune System Goes Astray. J Immunol 197, 3755-3761 (2016).
5. Claria, J., et al. Systemic inflammation in decompensated cirrhosis: Characterization and role in acute-on-chronic liver failure. Hepatology 64, 1249-1264 (2016).
6. Moreau, R., et al. Acute-on-chronic liver failure is a distinct syndrome that develops in patients with acute decompensation of cirrhosis. Gastroenterology 144, 1426-1437, 1437 e1421-1429 (2013).
7. Morel, E., et al. Autophagy: A Druggable Process. Annu Rev Pharmacol Toxicol 57, 375-398 (2017).
8. Deretic, V., Saitoh, T. & Akira, S. Autophagy in infection, inflammation and immunity. Nat Rev Immunol 13, 722-737 (2013).
9. Heckmann, B. L., Boada-Romero, E., Cunha, L. D., Magne, J. & Green, D. R. LC3-Associated Phagocytosis and Inflammation. J Mol Biol 429, 3561-3576 (2017).
10. Sil, P., Muse, G. & Martinez, J. A ravenous defense: canonical and non-canonical autophagy in immunity. Curr Opin Immunol 50, 21-31 (2018).
11. Sanjuan, M. A., et al. Toll-like receptor signalling in macrophages links the autophagy pathway to phagocytosis. Nature 450, 1253-1257 (2007).
12. Martinez, J., et al. Molecular characterization of LC3-associated phagocytosis reveals distinct roles for Rubicon, NOX2 and autophagy proteins. Nat Cell Biol 17, 893-906 (2015).
13. Yang, C. S., et al. Autophagy protein Rubicon mediates phagocytic NADPH oxidase activation in response to microbial infection or TLR stimulation. Cell Host Microbe 11, 264-276 (2012).
14. Matsunaga, K., et al. Two Beclin 1-binding proteins, Atg14L and Rubicon, reciprocally regulate autophagy at different stages. Nat Cell Biol 11, 385-396 (2009).
15. Zhong, Y., et al. Distinct regulation of autophagic activity by Atg14L and Rubicon associated with Beclin 1-phosphatidylinositol-3-kinase complex. Nat Cell Biol 11, 468-476 (2009).
16. Denaes, T., et al. The Cannabinoid Receptor 2 Protects Against Alcoholic Liver Disease Via a Macrophage Autophagy-Dependent Pathway. Sci Rep 6, 28806 (2016).
17. Lodder, J., et al. Macrophage autophagy protects against liver fibrosis in mice. Autophagy 11, 1280-1292 (2015).
18. Petiot, A., Ogier-Denis, E., Blommaart, E. F., Meijer, A. J. & Codogno, P. Distinct classes of phosphatidylinositol 3′-kinases are involved in signaling pathways that control macroautophagy in HT-29 cells. J Biol Chem 275, 992-998 (2000).
19. Kim, Y. R., et al. Peptide inhibition of p22phox and Rubicon interaction as a therapeutic strategy for septic shock. Biomaterials 101, 47-59 (2016).
20. Sanchez-Martin, P. & Komatsu, M. p62/SQSTM1—steering the cell through health and disease. J Cell Sci 131(2018).
21. Kim, J., Kundu, M., Viollet, B. & Guan, K. L. AMPK and mTOR regulate autophagy through direct phosphorylation of Ulkl. Nat Cell Biol 13, 132-141 (2011).
22. Tanaka, S., et al. Rubicon inhibits autophagy and accelerates hepatocyte apoptosis and lipid accumulation in nonalcoholic fatty liver disease in mice. Hepatology 64, 1994-2014 (2016).
23. Gandoura, S. & Moreau, R. Gene- and exon-expression profiling reveals an extensive LPS-induced response in immune cells in patients with cirrhosis. J Hepatol 58, 936-948 (2013).
24. Hogarth, P. M. Fc receptors are major mediators of antibody based inflammation in autoimmunity. Current opinion in immunology 14, 798-802 (2002).
25. Tomasi, T. B., Jr. & Tisdale, W. A. Serum Gamma-Globulins in Acute and Chronic Liver Diseases. Nature 201, 834-835 (1964).
26. Ben Mkaddem, S., et al. Shifting FcgammaRIIA-ITAM from activation to inhibitory configuration ameliorates arthritis. J Clin Invest 124, 3945-3959 (2014).
27. Mkaddem, S. B., et al. Lyn and Fyn function as molecular switches that control immunoreceptors to direct homeostasis or inflammation. Nat Commun 8, 246 (2017).
28. Bournazos, S., Hart, S. P., Chamberlain, L. H., Glennie, M. J. & Dransfield, I. Association of FcgammaRIIa (CD32a) with lipid rafts regulates ligand binding activity. J Immunol 182, 8026-8036 (2009).
29. Barabe, F., et al. Early events in the activation of Fc gamma RITA in human neutrophils: stimulated insolubilization, translocation to detergent-resistant domains, and degradation of Fc gamma RIIA. J Immunol 168, 4042-4049 (2002).
30. Walker, B. A., et al. Signal transduction events and Fc gamma R engagement in human neutrophils stimulated with immune complexes. J Immunol 146, 735-741 (1991).
31. Aloulou, M., et al. IgG1 and IVIg induce inhibitory ITAM signaling through FcgammaRIII controlling inflammatory responses. Blood 119, 3084-3096 (2012).
32. Levinsky, R. J., Cameron, J. S. & Soothill, J. F. Serum immune complexes and disease activity in lupus nephritis. Lancet 1, 564-567 (1977).
33. Nagelkerke, S. Q. & Kuijpers, T. W. Immunomodulation by IVIg and the Role of Fc-Gamma Receptors: Classic Mechanisms of Action after all? Front Immunol 5, 674 (2014).
34. Habib, A., et al. Inhibition of monoacylglycerol lipase, an anti-inflammatory and antifibrogenic strategy in the liver. Gut (2018).
35. Ma, J., Becker, C., Lowell, C. A. & Underhill, D. M. Dectin-1-triggered recruitment of light chain 3 protein to phagosomes facilitates major histocompatibility complex class II presentation of fungal-derived antigens. J Biol Chem 287, 34149-34156 (2012).
36. Tam, J. M., et al. Dectin-1-dependent LC3 recruitment to phagosomes enhances fungicidal activity in macrophages. J Infect Dis 210, 1844-1854 (2014).
37. Gomez, C. P., Tiemi Shio, M., Duplay, P., Olivier, M. & Descoteaux, A. The protein tyrosine phosphatase SHP-1 regulates phagolysosome biogenesis. J Immunol 189, 2203-2210 (2012).
38. Seifert, L., et al. Dectin-1 Regulates Hepatic Fibrosis and Hepatocarcinogenesis by Suppressing TLR4 Signaling Pathways. Cell Rep 13, 1909-1921 (2015).
39. Martinez, J., et al. Noncanonical autophagy inhibits the autoinflammatory, lupus-like response to dying cells. Nature 533, 115-119 (2016).
40. Carl, D. E., et al. A model of acute kidney injury in mice with cirrhosis and infection. Liver Int 36, 865-873 (2016).
41. Balasubramaniyan, V., et al. Importance of Connexin-43 based gap junction in cirrhosis and acute-on-chronic liver failure. J Hepatol 58, 1194-1200 (2013).
42. Teixeira-Clerc, F., et al. CB1 cannabinoid receptor antagonism: a novel strategy for the treatment of liver fibrosis. Nature Medicine 12, 671-676 (2006).
43. Hegde, P., et al. Mucosal-associated invariant T cells are a profibrogenic immune cell population in the liver. Nat Commun 9, 2146 (2018).
44. Colas, C., et al. An improved flow cytometry assay to monitor phagosome acidification. J Immunol Methods 412, 1-13 (2014).
45. Dandah, A., et al. Mast cells aggravate sepsis by inhibiting peritoneal macrophage phagocytosis. J Clin Invest 124, 4577-4589 (2014).

1. A method of treating sustained inflammation in a patient suffering from a chronic liver disease comprising administering to the patient a therapeutically effective amount of an agent capable of inducing LC3-associated phagocytosis.

2. The method of claim 1 wherein the patient suffers from cirrhosis.

3. A method of preventing acute-on-chronic liver failure in a patient suffering from cirrhosis comprising administering to the patient a therapeutically effective amount of an agent capable of inducing LC3-associated phagocytosis.

4. The method of claim 1 wherein the agent is a ligand of FcγRIIA.

5. The method of claim 4 wherein the ligand has an Fc region.

6. The method of claim 5 wherein the ligand is an immunoglobulin.

7. The method of claim 4 wherein the ligand is an anti-FcγRIIA F(ab′)2 fragment.

8. The method of claim 1 further comprising administering to the patient a therapeutically effective amount of IVIG intravenous immunoglobulin (IVIG).

9. The method of claim 2 wherein the cirrhosis is or is caused by alcoholic liver cirrhosis, primary biliary cirrhosis (PBC), liver fibrosis, chronic hepatitis, chronic autoimmune hepatitis, chronic alcoholic hepatitis, non-alcoholic steatohepatitis (NASH), A, B, C, D, E or G viral hepatitis, toxic metabolic liver damage, or fatty liver.
